(19) United States
(12) Patent Application Publication
(10) Pub. No.: US 2007/0093855 A1
(43) Pub. Date: Apr. 26, 2007

(54) Automaticaly operated endoscopic ligating instrument

(76) Inventor: Rugang Zhang, Beijing (CN)

Correspondence Address: Isaac A. Angres, Suite 301, 2001 Jefferson Davis Highway, Arlington, VA, 22202, US

(21) Appl. No.: 11/586,712

(22) Filed: Oct. 26, 2006

Related U.S. Application Data
(60) Provisional application No. 60/730,001, filed on Oct. 26, 2005.

(51) Int. Cl. A61B 17/10

(52) U.S. Cl. 606/140

(57) ABSTRACT

A double-tubed instrument for endoscopic ligation is provided with two separate tubes with bands and a piston next to the last band mounted on the internal tube. The internal tube with bands and piston is put inside of the external tube; a flexible cone-shaped tubular adapter secured both tubes as a tubular instrument at one end, which connects the instrument at the inserting end of the endoscope. The piston moves freely between the tubes and secures completely a sealed space under from the cone-shaped tubular adapter creating a hydraulic tank between the piston and the cone-shaped tubular adapter, which is connected with a conduit for conveying liquid in and stores the liquid serving as the driving force to move bands forward and dislodge the aimed elastic band one at a time to effect the ligation of multiple lesions during a single insertion of the endoscope.

This application claims the priority benefit under 35 U.S.C. section 119 of U.S. Provisional Patent Application No. 60/730,001 entitled “Automaticaly Operated Endoscopic Ligating Instrument” filed Oct. 26, 2006, which is in its entirety herein incorporated by reference.

### BACKGROUND OF INVENTION

The endoscopic treatment of lesions presently encompasses a variety of techniques such as eletrocauterization, laser photocoagulation, heat therapy by the application of heat probes, and scelerotherapy which involves the injection of medicine into a target varix by a needle passed through the working channel of the endoscope. A further, widely used and increasingly promising technique involves the ligation of lesions, wherein mucosal and submucosal tissue is strangulated by an elastic ligature.

A variety of instruments for effecting the ligation of body tissue by the application of an elastic ring are well known in the prior art. Some of these instruments, because of their rigidity and size are suited only for treatment of lesions that are in the external revisions of the body or in the shallow body cavities. Others are particularly suitable as for tubal ligation, when the abdominal cavity has been opened surgically.

U.S. Pat. No. 3,760,810 to Van Hoorn discloses an endoscope-equipped instrument comprising a devise with two tubes mounted one inside the other, with the inner tube protruding at the front of the outer tube. Means are included to move the outer tube forwardly relative to the inner tube and cause an elastic cord to be dislodged and placed about the tissue to be legated. In U.S. Pat. No. 4,257,419, there is disclosed an instrument for ligating hemorrhoids wherein a suction tube fitted inside a proctoscope provides means for sucking the hemorrhoid into a suction cavity where a ligating ring is applied. Both of these instruments are rigid devices suited for treating lesions closed to the external regions, and both are equipped with only a single elastic ring for treating a single lesion.

There are also instruments in the prior art which employ laparoscope-assisted means for ring ligation such as shown in U.S. Pat. Nos. 4,257,420 and 4,471,766, wherein the instruments are each equipped with a single elastic ring and utilize forceps to position the tissue for ring ligation.

In U.S. Pat. No. 3,870,048, there is disclosed a ring applicator device having forceps slidably mounted in a cylinder for grasping the fallopian tube and including means for displacing an elastic ring to effect with a plurality of elastic bands, but its rigidity precludes its use with a flexible endoscope for treating the deeper regions for an internal organ, such as the alimentary tract.

A flexible endoscopic instrument used for ligation purpose and disclosed in U.S. Pat. No. 4,735,194 comprises a flexible fiber optic endoscope on the end of which is secured an outer tube and an inner tube reciprocally movable therein. A trip wire is fastened to the inner tube to provide rearward motion to the inner tube to case an elastic ring fitted about the inner tube to slide off and effect ligation. While this instrument is suitable for ligating lesions deep within the alimentary tract, it can only be used to treat one lesion during a single insertion of the instrument.

U.S. Pat. No. 5,624,453 discloses a flexible endoscopic instrument provided with a plurality of elastic rings mounted on one tube of a pair of tubular members which are affixed in coaxial relation to the insertion end of an endoscope. Although the instrument is more flexible compared to the previous ones for treating lesions closed to the external regions, and equipment with multiple elastic rings for treating internal lesion, it is operated manually and requires a high level of skill of human operators during the operation, and the actuating cable providing the driving force of motion of the instrument sometimes stands in front of the illumination device of the endoscope which blocks the view of the doctor during the operation.

In the past few years, the endoscopic ligation instrument has not been widely used in the treatment of lesions present in human organs, such as the esophagus, stomach or colon because of the complexity of the instrument and the skills required by the operator. When an endoscopic instrument is equipped with manually operated actuating cables, the operation of the instrument depends on skills of the operating doctor. Also the structure of the actuating cable in the instrument is more complicated and the actuating cable can stand in front of the viewing device blocking the view of the doctor operating the instrument. In addition to the blocked view, the momentary lost of the time during an operation also caused concerns. The instrument, when inserting the elastic ring to the lesion, it has to be aimed at the lesion precisely, especially in the situation of large quantity of internal bleeding cases. With the actuating cable floating in front of the viewing mirror, a doctor could miss the target lesion and thus lose precious time during the procedure for the intended treatment.

### SUMMARY OF THE INVENTION

A double tubed endoscopic instrument is provided with a plurality of elastic ligating bands and piston mounted on the internal tube of tubular members which are affixed in coaxial relation to the insertion end of an endoscope. The bands are adapted to be dislodged therefrom in sequence at selectively controlled times for treating multiple lesions. The endoscope is equipped with illumination and viewing means to facilitate orientation of the instrument in the body organ, and longitudinally extending tubular passages comprising a channel through which objects may be passed and suction applied for drawing the lesion tissue into the tubular end of the endoscope to facilitate ligation of the lesion. A hydraulic tank is equipped together with the instrument for providing a driving force for the bands' motion on the internal tube to sequentially dislodge the elastic bands from the instrument at controlled times. Each of the elastic bands can be dislodged from the endoscope and placed in ligating relation to a lesion when lesion tissue is drawn into the innermost of the tubular channel by a suction force applied through the suction channel of the endoscope and each of the bands can be applied to a different one of the multiple lesions in the body organ during a single insertion of the endoscope.

In one embodiment of the invention, a double-tubed instrument is provided with two separate tubes with bands and a piston next to the last band mounted on the internal tube. The internal tube with bands and piston is put inside of the external tube. A flexible cone-shaped tubular adapter secures both tubes as a tubular instrument at one end, which connects the instrument at the inserting end of the endoscope. The piston moves freely between the tubes and secures completely a sealed space under the cone-shaped tubular adapter creating a hydraulic tank between the piston and the cone-shaped tubular adapter, which is connected with a conduit for conveying liquid in and stores the liquid serving as the driving force to move bands forward and dislodge the aimed elastic band one at a time to effect the ligation of multiple lesions during a single insertion of the endoscope.

In a second embodiment, a conduit is connected with the hydraulic tank between the internal and external tubes in order to provide a fluid such as a liquid slected from the group consisting of purified water, sterilized water or physiological saline solution into the hydraulic tank as the means for providing the driving force to move the elastic bands to the position for dislodgement. After the dislodgement of the elastic band, the driving force can bring the band next at the end of the internal tube back to its original position from the dislodging position to void an unaimed dislodgement. The other end joint of the conduit is connected to the hydraulic injector of the hydraulic injection device, which provides the liquid to the hydraulic tank through the conduit. Liquid shall be pulled into the hydraulic injector first, then, pushed into the hydraulic tank through the conduit. The driving force of the instrument is given by an actuating motor through the hydraulic injector.

In a third embodiment, an automatically controlled ligating positioner is connected with the hydraulic injection device to control the driving force to be provided to ligate the elastic bands to be adapted to the position for dislodging. The automatic control is done by a preprogrammed control circuit and a ligating positioner, which functions to indicate the hydraulic injection device to provide the calculated amount of liquid to put the elastic band to be adapted at position to be dislodged. The ligating positioner is comprised of a positioning cone array, infrared sensor, wire, the power supply and control circuit. Operation is as follows: holding the hydraulic endoscopic ligating instrument at a 45° angle, connecting the conduit and the hydraulic injector, pushing liquid into the hydraulic tank through the conduit and forcing the air inside of the hydraulic tank out through a groove, or other means to force the air out, insert the ligating positioner at the innermost end of the internal tube, press the aiming button on the control panel to start the hydraulic injector for sending liquid to move the piston, thereby, ligating the elastic bands to the position for dislodgement. The infrared sensor then signals the hydraulic injector to stop working when the elastic band reaches the position adapted to dislodge, thus completing the dislodgement preparation. Take off the ligating positioner and reconnect the ligating instrument and the instrument to effect the ligation of a lesion.

In a fourth embodiment, a hydraulic injection device, together with the ligating positioner, provided with related mechanical components, control circuit, actuating motor, power supply and operating control panel. The hydraulic injection device dislodges the elastic bands precisely on the lesions. The control circuit of the hydraulic injection device is a programmable circuit used for giving dislodging signals to the actuating motor. The data for the calculated amount of the liquid needed to move the elastic bands for ligation dislodgement, the angle and number of the rotation of actuating motor for hydraulic injector to provide the needed liquid to the hydraulic tank for pushing the elastic bands to the position of adapting for dislodgement are preprogrammed, and data for positioning of ligating positioner of the elastic bands from the infrared sensor shall all be programmed into the control circuit. Thus, when the doctor observes the suction of the lesion into the tubular member of the endoscope, he presses the dislodging button, the actuating motor will rotate at the programmed rate to drive the pushing pole to the hydraulic injector for the programmed amount of liquid, just enough for the elastic band to be adapted for ligation dislodgement from the instrument. Operation buttons for each function of the system were designed for the convenience and the precise control of the instrument.

In a fifth embodiment, an automatically operated hydraulic endoscopic instrument for ligating a multiplicity of lesion within a hollow body organ, such as the alimentary tract, is disclosed. The instrument comprises a double tube system with elastic bands mounted on the internal tube driven by a hydraulic injector operated automatically by a preprogrammed circuit control system. The instrument is attached to an endoscope having a forward insertion end and a rearward end. The endoscope includes means for illumination and viewing through said endoscope, means for providing a suction force at said insertion end, and a working channel. A tubular member having a forward distal end, a rearward end and a longitudinal axis. The rearward end has means for providing attachment to the insertion end of the endoscope. A plurality of ligating elastic bands are removably mounted one by one on the internal tube of the double tube hydraulic endoscopic instrument in coaxial relation thereto and each of said bands being pushed by the programmed hydraulic power along the internal tube to be dislodged in controlled sequence during a single insertion of the endoscope. The ligation of the lesions inside of the human body is done by the elastic bands in the rectangular shape for the grasp of the human organ to avoid the easy dropping. The hydraulic injector provides the moving force to push the elastic bands mounted on the internal tube of the instrument to be dislodged and is operated by the preprogrammed control circuit installed with the hydraulic injection device. The hydraulic injector is connected by a conduit to the instrument as the media to convey the hydraulic power to the instrument. The hydraulic injector is driven by an actuating motor at a programmed rotation ratio to insure providing the needed amount of liquid to push the elastic bands to be dislodged from the instrument. A ligating positioner with an infrared sensor is also used with the instrument to measure the needed liquid as driving force to push the elastic bands to be dislodged. Said ligating positioner provides the instruction to the control panel, which then instructs the actuating motor to run for the calculated amount of the liquid for the hydraulic injector to convey to the ligating instrument.

The word band in the present specification is intended to include elastic bands and elastic rings. The elastic bands may be of any shape i.e., rectangular.

### BRIEF DESCRIPTION OF THE DRAWINGS

FIG. 1 is a general layout of the automatically operated hydraulic endoscopic ligating instrument, which comprises three major parts: endoscopic ligating instrument, a ligating positioner and the hydraulic injector, representing a preferred embodiment of the invention.

FIG. 2 is a longitudinal view, partly in section of the hydraulic endoscopic ligating instrument representing one of the three major parts of the invention.

FIG. 3 is a longitudinal section view of the hydraulic endoscopic ligating instrument showing each elements of the instrument within.

FIG. 4 is a frontal plan view of the insertion end of the hydraulic endoscopic ligating instrument of FIG. 2.

FIG. 5 is a section view of showing the two tubes connected by a piston with the opening for the conduit between two tubes.

FIG. 6 is a perspective view of a further embodiment of the present invention.

FIG. 7 is a section view of the cone-shaped tubular adapter showing as the number 7 in FIG. 2, which connects the hydraulic ligating instrument to the endoscope.

FIG. 8 is a plain view of the band mounted on the internal tube shown in FIG. 6 and FIG. 3.

FIG. 9 is a plain view of the hydraulic injector as taken along the line 16-16 in FIG. 18 which pushes the liquid through the conduit to the hydraulic endoscopic ligating instrument as the driving force.

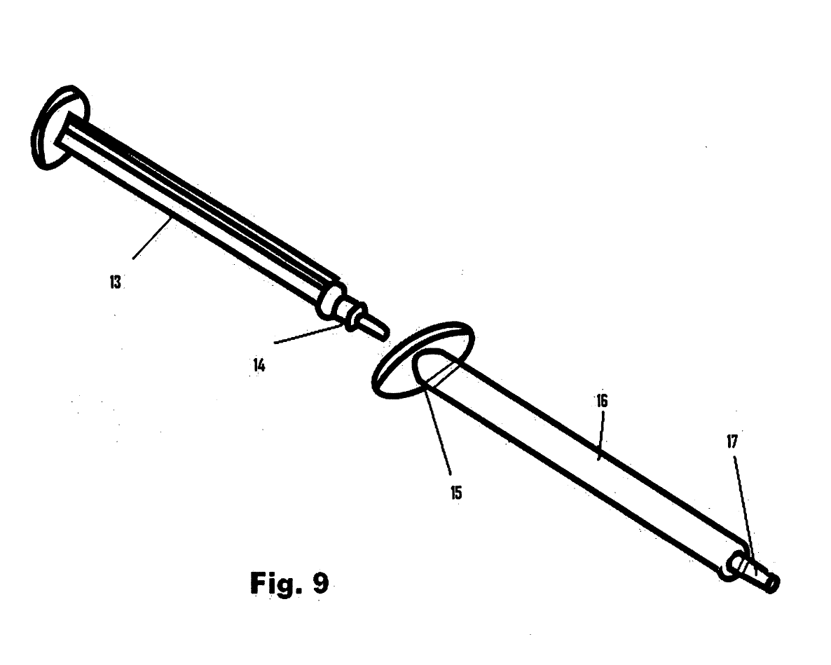

FIG. 10 is a plain view of an embodiment of the invention as illustrated in FIGS. 2 through 9.

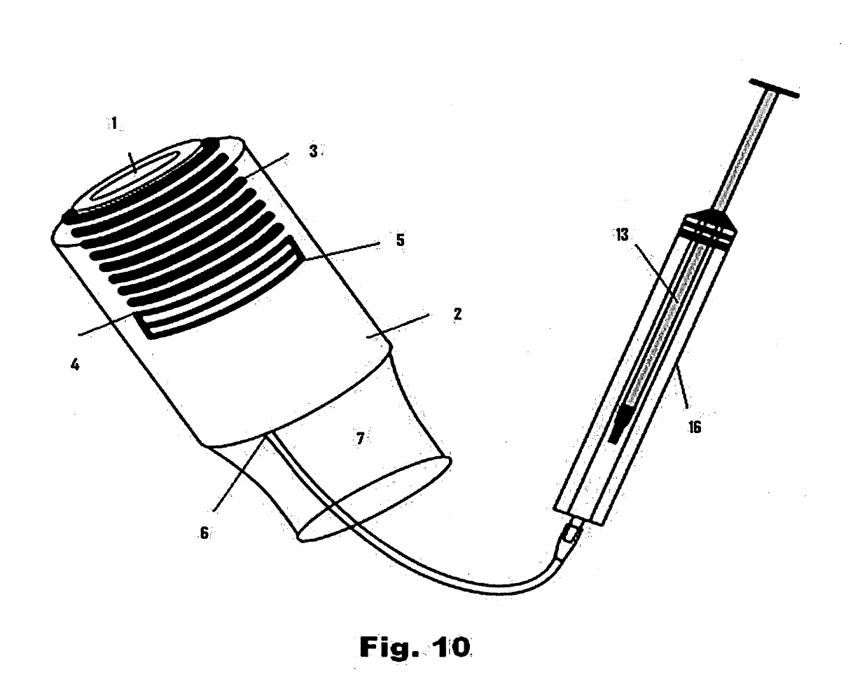

FIG. 11 is a fragmentary view, partly in section, showing the insertion end of the instrument of the invention applied to a lesion with lesion tissue drawn by suction into an inner internal tube on the end of the instrument.

FIG. 12 is a fragmentary view, partly in section, showing an elastic ligating band applied about a lesion after its dislodgement from the end of the hydraulic endoscopic instrument of the invention.

FIG. 13 shows a lesion with an elastic band applied in strangulating relationship hereto.

FIG. 14 is a section view of the hydraulic endoscopic ligating instrument showing the direction of the driving hydraulic power, which pushes the ligating bands forward to be dislodged.

FIG. 15 is a similar section view of the instrument showing the due directions of the driving hydraulic power, which moves the ligating bands forward to be dislodged and backward after the dislodgement.

FIG. 16 is a section view of the piston, which seals the space between internal tube and external tube creating a hydraulic tank shown as no. 4 in FIG. 3.

FIG. 17 is a general layout of the automatically operated hydraulic ligating instrument indicating the parts and sections as in FIG. 1.

FIG. 18 is a plain view of layout of the hydraulic injector device as shown in FIG. 17, number 39.

FIG. 19 is a plain view of the layout of the hydraulic of the control panel of the hydraulic injection device, which controls the functions of the hydraulic injection device.

FIG. 20 is a plain view of an embodiment of the device by which means for the hydraulic power needed provides the liquid required for the ligating band to be dislodged.

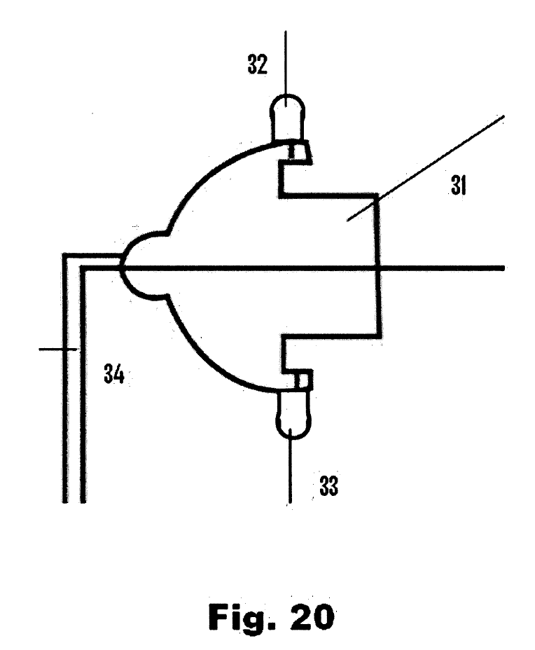

FIG. 21 is a plain view of the actuating motor as numbered 40 in FIG. 18 which provides the driving force to push the liquid into the instrument.

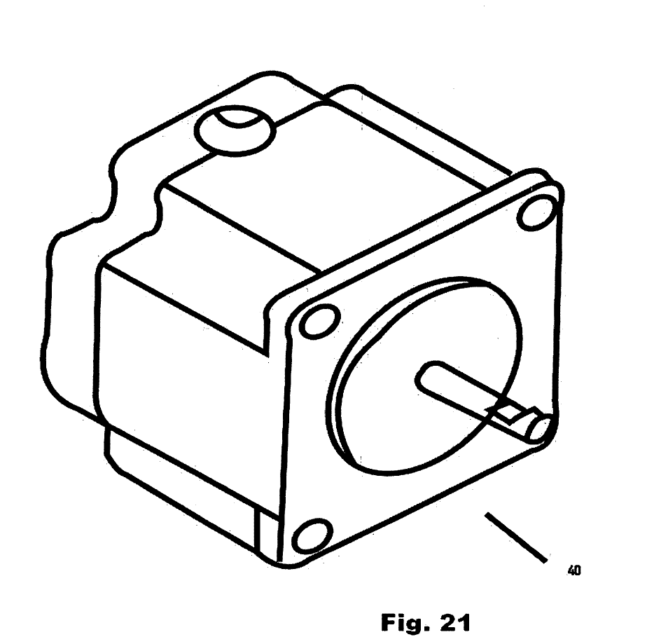

FIG. 22 is a plain view of the a part to fix the hydraulic injection device.

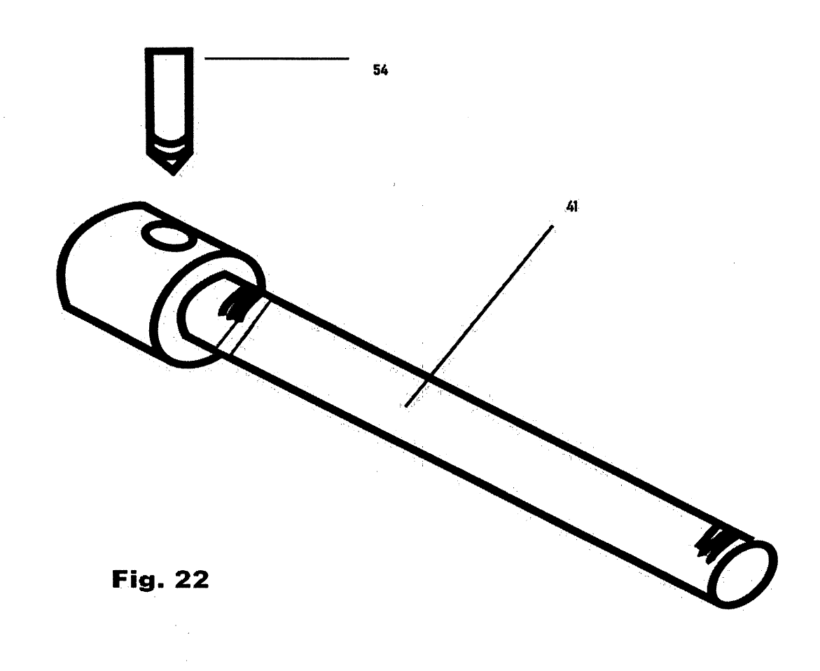

FIG. 23 is a plain view of a part used to fix the hydraulic injection device in FIG. 18.

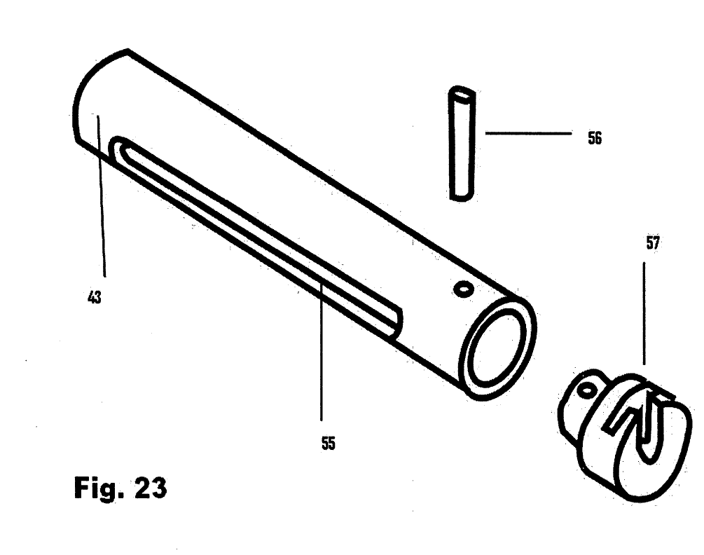

### DESCRIPTION OF THE PREFERRED EMBODIMENTS

For the purpose of promoting an understanding of the principles of the invention, reference will now be made to the embodiment illustrated in the drawings and specific language will be used to describe the same. It will nevertheless be understood that no limitation of the scope of the invention is thereby intended. Such alternations and further modifications in the illustrated device, and such further applications of the principles of the inventions as illustrated therein being contemplated as would normally occur to one skilled in the art to which the invention relates.

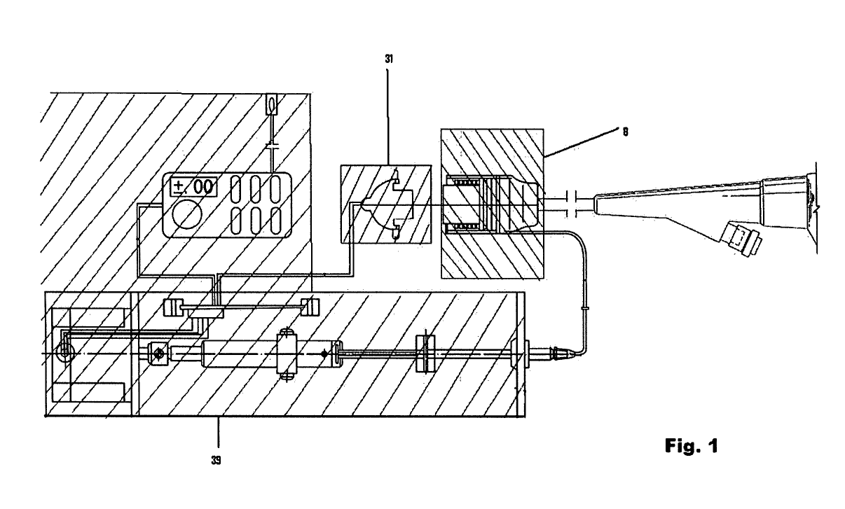

Referring more particularly to the drawings, there is shown in FIG. 1 a hydraulic endoscopic instrument 8 of a length which permits access to the deeper regions of a hollow body organ, such as the ulcer in the stomach. The instrument 8 comprises a conventional endoscope with an operating controls section of rigid construction and a flexible section which extends therefrom and is of a length sufficient to reach the deeper regions of the ulcer in the stomach. The distal end of the flexible section is the insertion end of the endoscope and the viewing end of the endoscope is at the end of the rigid operating controls section remote from the end thereof which connects to the flexible section.

The endoscope is provided with passages which extend longitudingally therein from its insertion end to exit ports near the viewing end of the endoscope. The passages comprise an illumination channel through which is inserted a fiber optic cable for the transmission of light from a light source, a viewing channel which is also provided with a fiber optic cable for viewing purposes, and a channel through which objects may be passed or suction applied. The illumination channel and suction channel exit laterally through a lateral extension of the side wall of the endoscope at a location on the operating controls section near the viewing end. At there exit location, the fiber optic cables from channel and channel are connectable through a servicing cable to a control device (not shown) which is adaptable for supplying illumination to the transmission optic cable and for connecting the cannel to an appropriate means for applying a suction therethrough. The fiber optic viewing channel extends to the viewing lens of the endoscope which may be provided with a viewing lens and an adapter for mounting a camera thereon, if desired.

The endoscope is also provided with a working channel which extends through the endoscope from its insertion end to an exit in the lateral extension. The working channel includes a branch which extends through an angularly extending protuberance to a section exit near the viewing end of the endoscope.

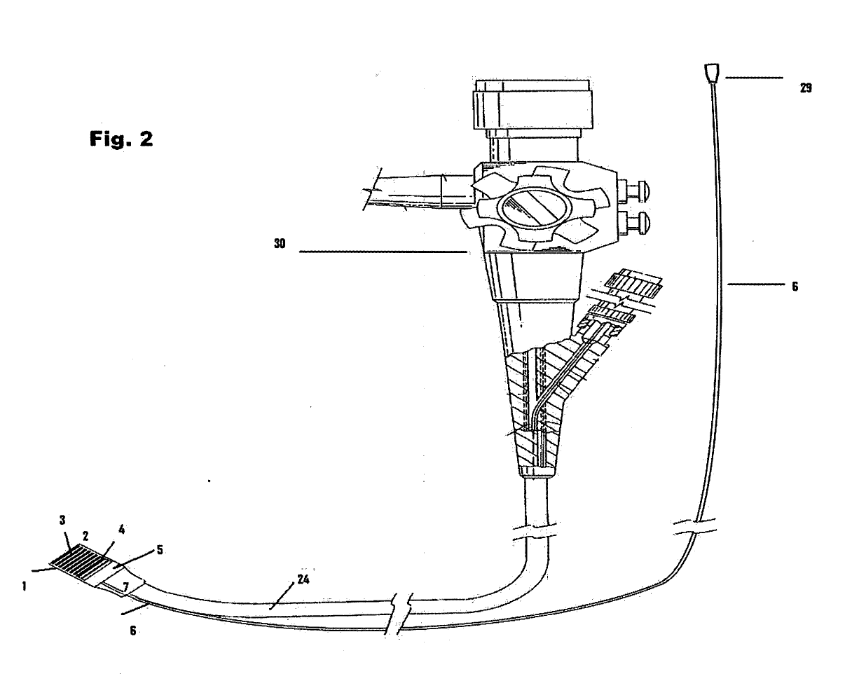

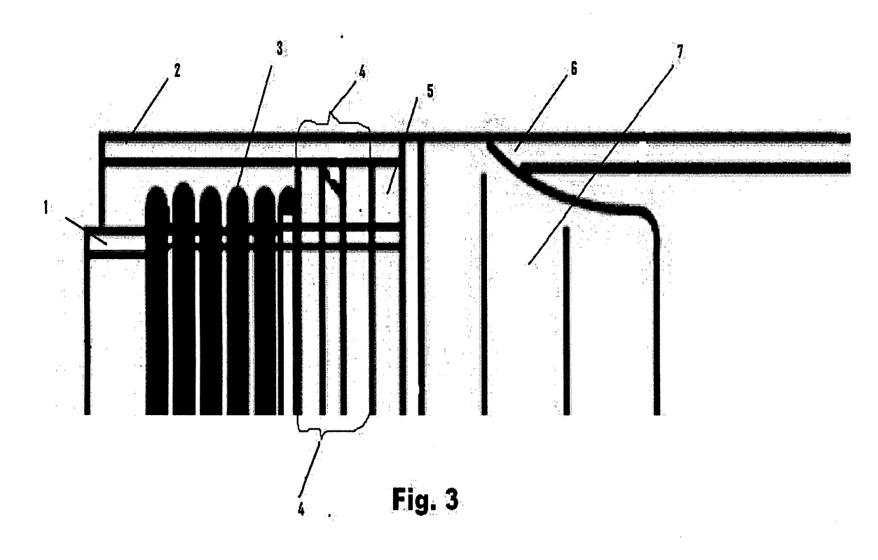

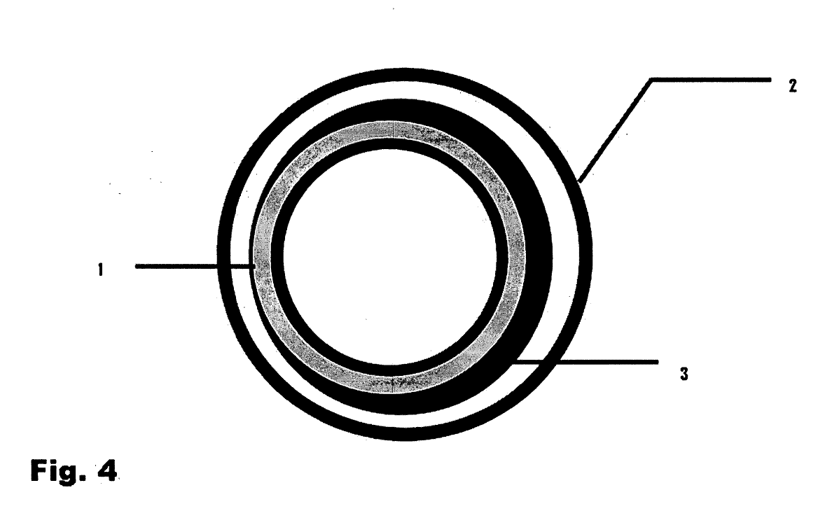

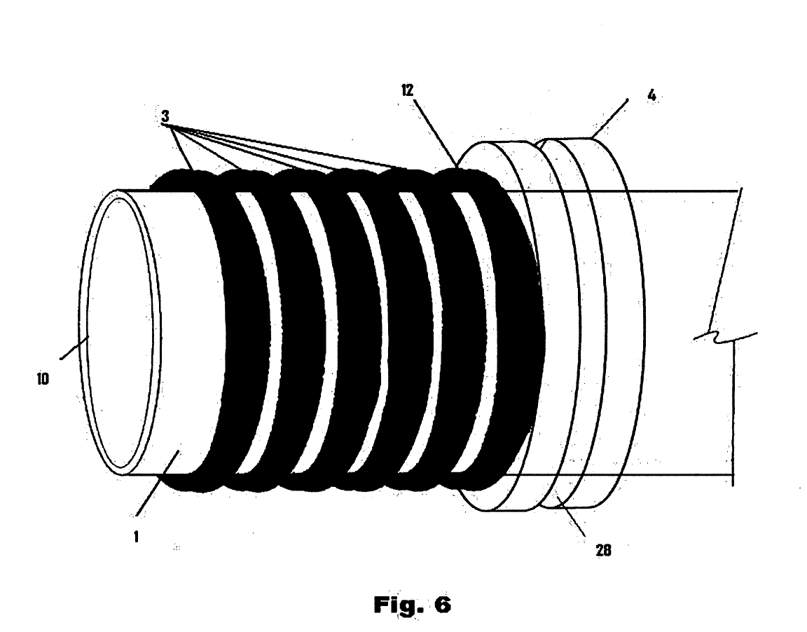

In the embodiment of the invention shown in FIG. 1 through 7, showing the first novel aspect of the invention, the insertion end of the endoscope is fitted with an assembly of coaxially arranged tubes 1, 2. The external tube 2 of which is secured in coaxial relation thereto by a cone-shaped soft material, preferably silica gel, solid tubular adapter 7, which provides a friction fit as shown in FIG. 2 to the insertion end of the endoscope. The tube 2 is preferably of transparent plastic material for enhancing the illumination and field of vision from the insertion end of the endoscope. The internal tube 1 of which is mounted with elastic bands one by the other along the tube and a piston next to the last band thereon which is inserted inside the tube 2. The internal tube 1, preferably of transparent plastic material as tube 2, is also secured in coaxial relation thereto by the same cone-shaped soft material solid tubular adapter 7 at the end as shown in FIG. 3, which is the connectable adapter to the distal end of the flexible section of the insertion end of the endoscope as shown in FIG. 2. The piston 4 placed next to the bands on the internal tube facilitates the complete sealing between the internal tube 1 and external tube 1, as the space between the two tubes underneath the piston 4 and above the connections of internal tube 1 and external tube 2 to the cone-shaped tubular adapter 7 as shown in FIG. 3 creates a space as the hydraulic tank 5 of which stores the liquid 22 providing the driving force to push the bands forward to be dislodged.

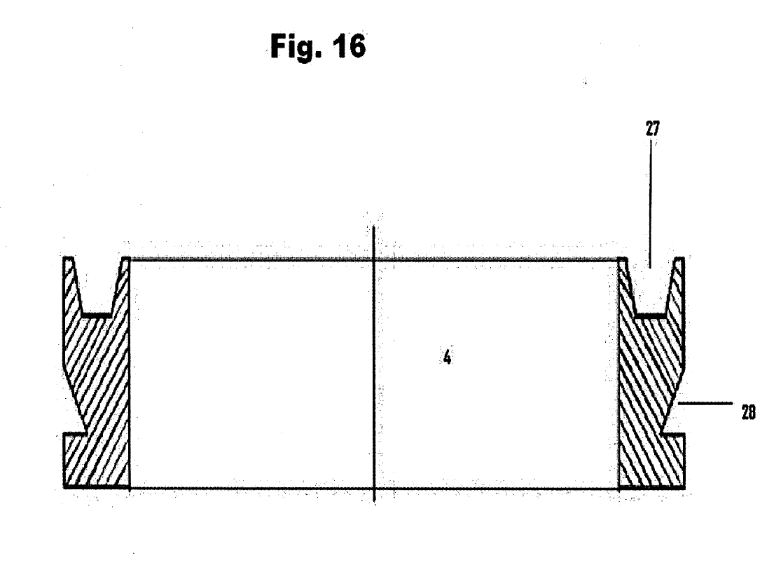

The hydraulic tank 5 between the two tubes as shown in FIG. 3 can be extended as more liquid is put in for moving the band forward and dislodgement. With the movement of the piston, the hydraulic tank becomes bigger and smaller. For the easy movement of the piston 4 and reduction of the resisting force on the inner and outer walls of the tubes, a fillister 27 on the top of the piston and a dovetail 28 at the side as shown in FIG. 16 are made, thus to improve the sealing and moving of the piston 4. The material for the piston 4 is preferably polytetrafluoroethylene, however it could be other suitable materials. The size of the piston is the same as the band.

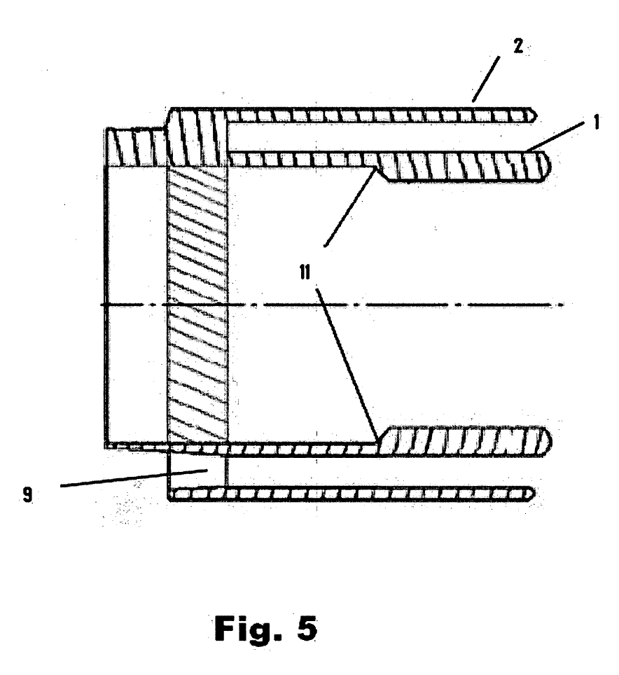

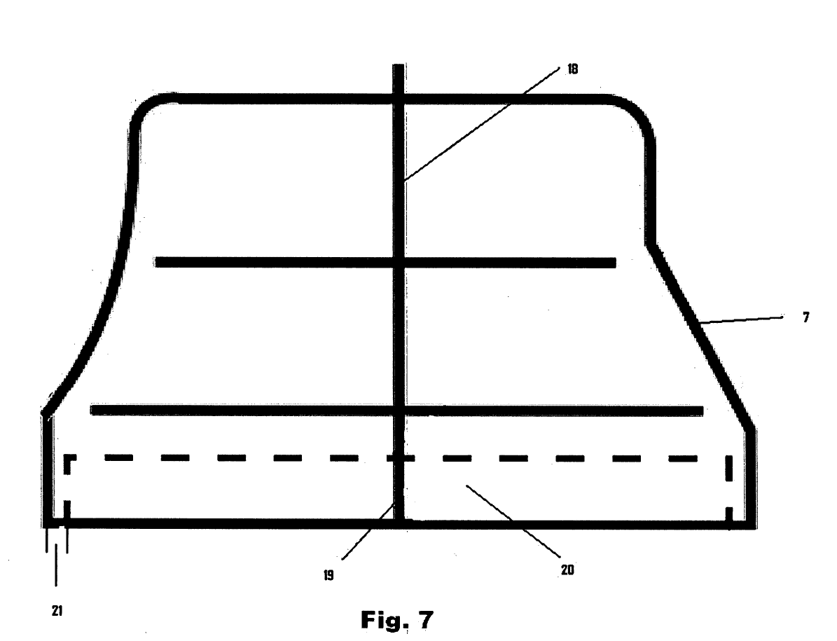

The cone-shaped soft tubular adapter 7, which connects and secures the tube 1 and tube 2 coaxially relation to the insertion end 24 of the endoscope 30, has a lock-bracket 19 around the open end as shown in FIG. 7, for tight fitness. The width of the lock-bracket is the thickness of the internal tube 1, which fits into the lock-bracket of the cone-shaped tubular adapter to secure the tightness of the connection between the internal tube 2 and the said adapter and the smooth inner wall at the connection of two parts, while the outer edge 21 at the open end of the said adapter blocks the external tube 2 from moving as shown in FIG. 3. The material for the said adapter is preferably silica gel, as the material provides the elasticity for the easy adjustment of the fitness of parts, and the slight roughness of the surface of the said adapter also facilitates the grasp between the instrument and the insertion end of the endoscope 30. The inner wall of the tube 1 comes in at 15° as shown in FIG. 5 at the lower half on the side of connecting the adapter, which fits the standard size of the insertion end 24 of the endoscope 30 blocking it from slipping further inner the instrument and the tight fitness of the instrument to the insertion end 24 of the endoscope 30.

An opening 9 at one point between the internal tube 1 and external tube 2 as shown in FIG. 5 provides the inlet and outlet for the liquid to the hydraulic tank 5. A narrow conduit 6 is connected to this opening 9 which conveys liquid 22 in and out of the hydraulic tank 5, the other end 29 of the long conduit is connected to the joint 17 of the hydraulic injection device 39 as shown in FIG. 2, which pushes in the liquid 22 through the conduit 6 to the hydraulic tank 5. The ideal length of the conduit is 4-6 feet or longer. The material for the conduit is preferably a transparent material.

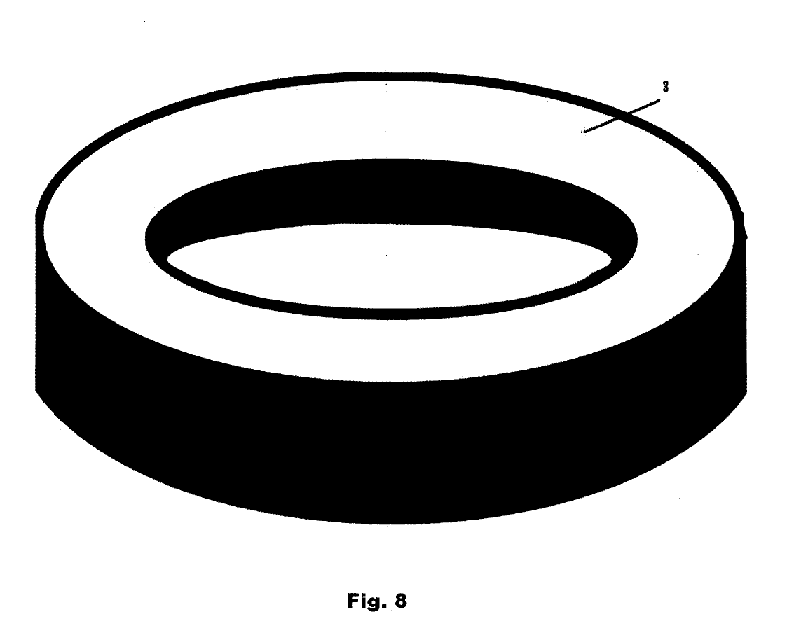

The elastic bands 3 used for ligation of lesions 21 inside the human organ mounted on the tube 2 are rectangular in shape as shown in FIG. 8 for the easy grasp of the human organ to avoid the dropping. The material for the band 3 is preferably natural rubber or other synthetic elastic materials. The size of all the bands 3 has to be precisely the same for the operation of the instrument and lubricant applied to the bands and the outer surface of the internal tube 1 to ease the movement of the bands 3. As the indictor of the last band 12 on the internal tube 1, the color of the last band on the tube is different from the others, thus to remind the human operator the last band available to avoid the possible accident caused by the dislodgement without a band during the ligation.

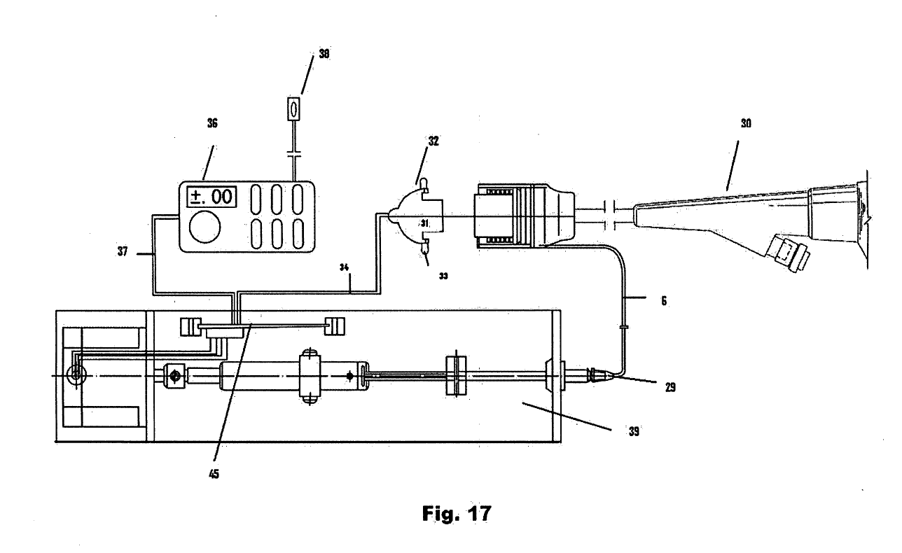

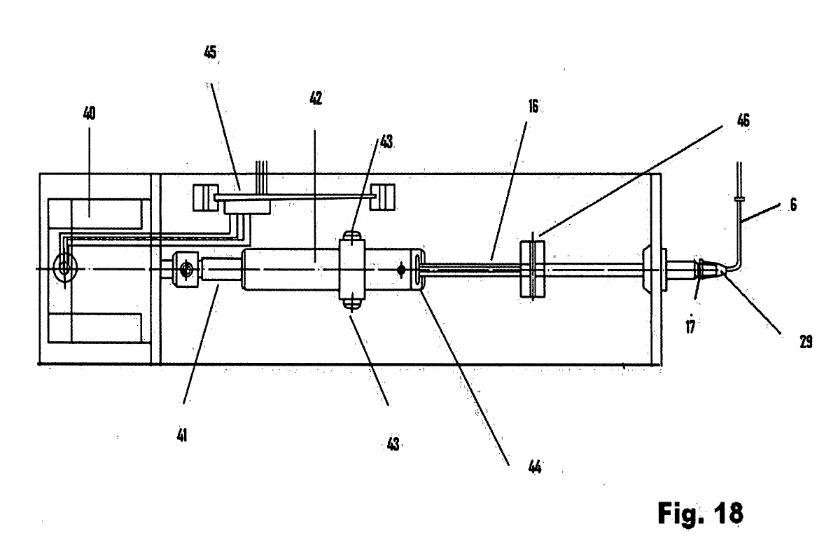

The novel technology shown in this invention is the change of the format of the driving force for the bands to be dislodged from the ligation instrument, which applies the hydraulic power and use the liquid 22 as the media to transmit the driving force. As shown in FIG. 17, the hydraulic injection device 39 for the whole instrument comprise a control panel 36, a actuating motor 41, hydraulic injector 16 of which is fixed in a box with an opening at one end for the connection of the joint to the conduit 6. A preprogrammed control circuit 45 (not shown) installed inside the control panel 36 of which controls the operation of the hydraulic injector 16. Said hydraulic injector 16 of which applies the working theory of the injector used in the hospital is connected to the conduit 6 at the joint 29 as shown in FIG. 17 to the instrument for conveying the hydraulic power to dislodge the band 3. The hydraulic injector 16 is driven by an actuating motor 40 in the device as shown in FIG. 18 at the programmed rotation rate to push a pushrod which in tern to move the hydraulic injector 16 for convening the calculated amount of liquid 22 into the instrument to move the elastic bands 3 to be dislodged from the instrument. Preprogrammed data for the operation of the hydraulic injection system are installed in the control circuit 45.

For the precise automatic operation of the hydraulic ligating instrument and successful ligation of each dislodgement of the band, a ligating positioner 31 which is a novel aspect of the invention, with an infrared sensor receiver 32 and infrared sender 33 is also used with the instrument to measure the precise amount liquid 22 needed as the driving force to move the elastic bands 3 to be dislodged. The infrared sensor 32, 33 is connected to the controlling circuit 45 within the hydraulic injection device 39 by a cable 37, and it shall be inserted at the utmost end of the instrument as shown in FIG. 2 when liquid 22 is already filled the hydraulic tank 5 and no air in the hydraulic tank 5. The hydraulic ligating instrument 8 shall be held at the angle of 45° as shown in FIG. 2 when the ligating positionor is applied for the precise reading. The human operator then press the positioning button 48 on the control panel 36, the said infrared sensors receiver 32 receive data when the ligating positioner inside of the instrument 8, sensor 33 then sends data to the control circuit based on the distance of the lesion inside the human organ, the control circuit 45 instructs the actuating motor to start working for the calculated amount of the liquid for the hydraulic injector 16 to convey the liquid 22 to the ligating instrument 8 thus move band to the position at edge of the internal tube 10 for dislodgement. At this time, the infrared sensor 33 sends signal to the control circuit to stop the actuating motor. Thus the positioning of band 3 is completed. Take off the ligating positioner 31 from the instrument 8 and connect the instrument 8 with the insertion end 24 of the endoscope 30 for dislodgement. Using the illumination device of the endoscope to observe the insertion of the instrument in the human organ and suction of the lesion into the insertion end through the instrument, one then presses the buttons on the control panels for adjusting the position or dislodging to complete the dislodgement of the ligating band to lesions.

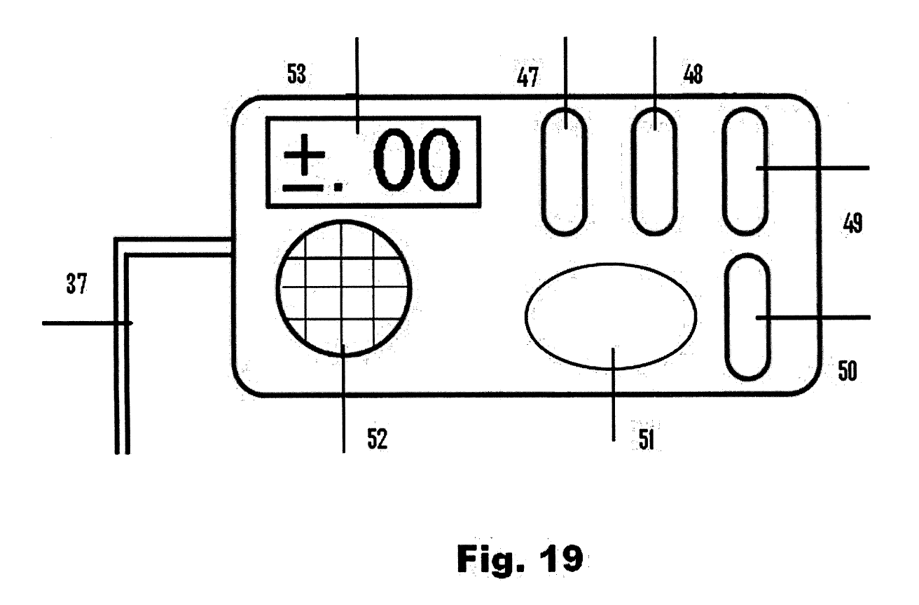

The control panel 36 of the hydraulic injection device 39 as in FIG. 19 comprise with a digital screen 53 showing data for each function to be run, a positioning button 48 for the hydraulic injection is to instruct the system to push enough liquid 22 to get the band to the position to be dislodged, a reset button 49 for adjusting the data memory for dislodge, a motor button 50 for adjusting the rotation rate and angle of the actuating motor, a dislodgement button 51 for instructing the instrument to dislodge the band to ligate the lesion, a returning button 47 for instruction to move the band to its original position before the dislodgement, and a buzzer 52 which will make a noise every time when the band 3 is positioned or the band 3 is dislodged.

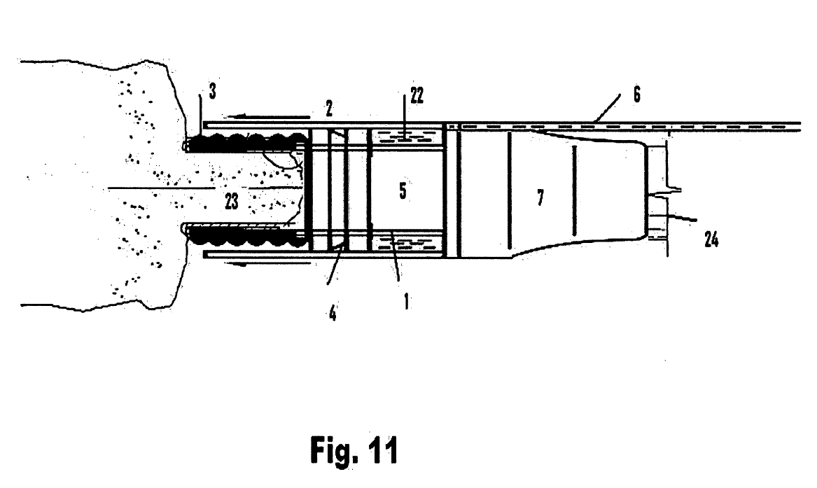

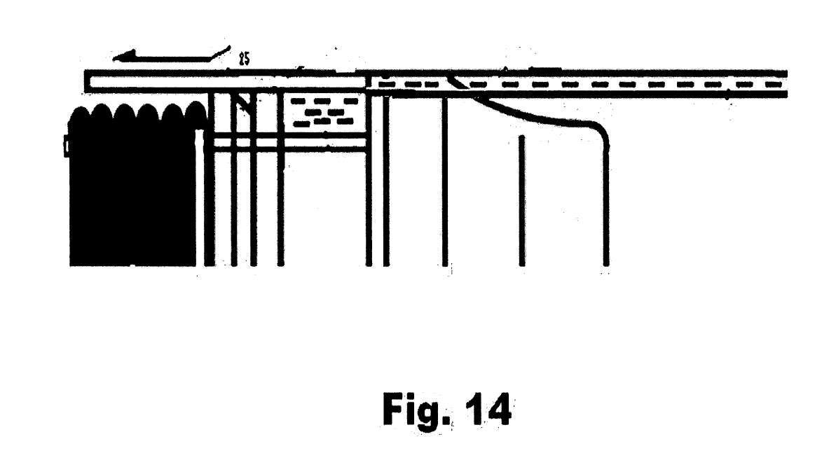

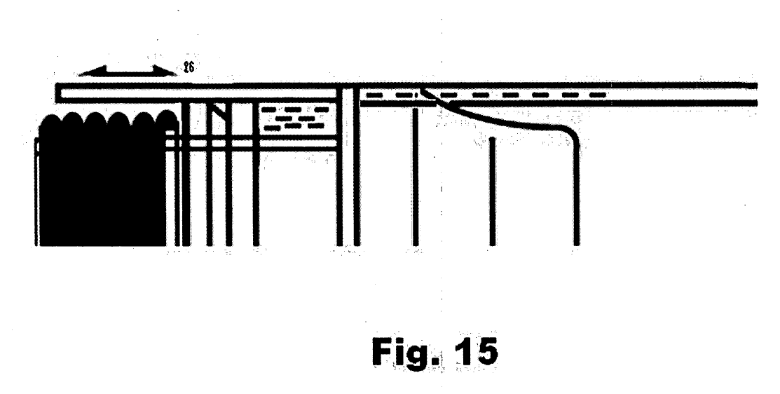

The hydraulic injection device 39 in the system controls the driving force to push the band forward 25 for the dislodgement as shown in FIG. 14, it can also instruct the driving force band backward 26 when the dislodgement is completed as shown in FIG. 15 to avoid the band falling off the end of the internal tube 10. When treating a patient, the hydraulic endoscopic instrument 8 of the invention is first introduced into the affected organ, such as an ulcer in the stomach, to place the insertion end of the endoscope in the vicinity of the lesion over the ulcer in the stomach. In some instance, however, it may be preferred that insertion of the instrument be preceded by the insertion of an endoscopic instrument into the ulcer in the stomach. The instrument is then oriented for sighting for a target lesion such as lesion 23 shown in FIG. 11, and the instrument advanced under the control of a human operator until the distal end of a double tubed instrument 8 contact the lesion area and is placed in surrounding relation to the target lesion.

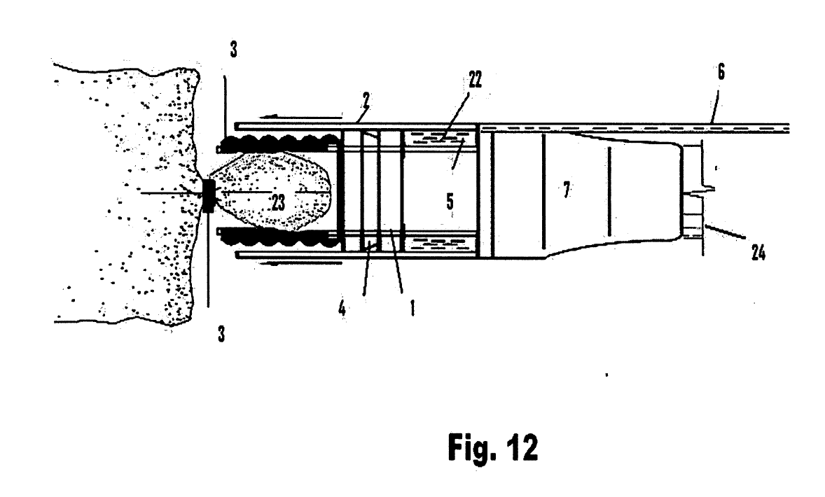

A suction force is then applied through the suction function of the endoscope to completely draw the lesion tissue into the inner tubular member as shown in FIG. 12.

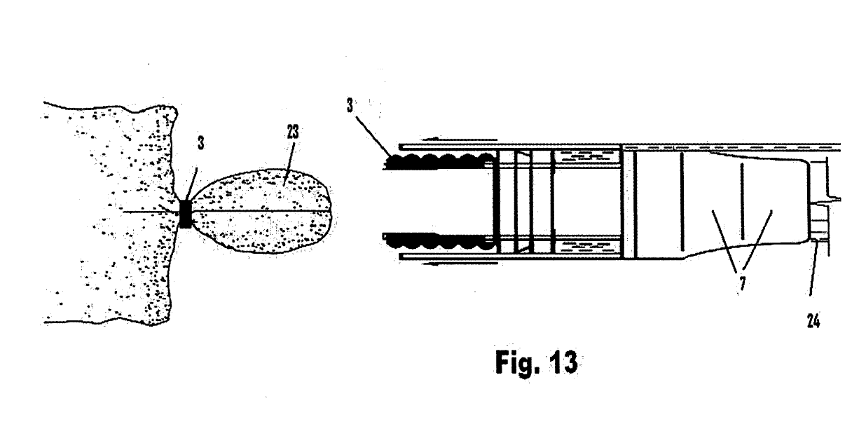

The operator then press the dislodgement button 51 on the controlling panel in FIG. 19, the control circuit then instructs the actuating motor to push the hydraulic injector to push the exact amount of liquid into the hydraulic tank that moves the piston to push the band to the position for dislodgement, then the human operator press the dislodgement button 51 on the control panel 36, the instrument repeats the operation again then the piston push the band to be dislodged from the instrument and placed in ligating relation about the base of the target lesion as shown in FIG. 12. The end of the instrument is then withdrawn from around the lesion tissue, as shown in FIG. 13.

The actuating motor of the hydraulic injection device is fixed on a big opening at the base of the hydraulic injection device, usually in a box, then connect the pushrod with the axle of the actuating motor by a cylindrical screw for pushrod The pushrod is then connected with the pushing bar of the hydraulic injector 13, 16 by a pitch. Thus the actuating motor coupled with the pushrod which in term push the hydraulic injector to send the liquid into the hydraulic tank in the instrument to dislodge the band.

For ease of identifying important elements of the invention, the following glossary is attached identifying what each number in the drawings denotes.
1. internal tube
2. external tube
3. elastic band
4. piston
5. hydraulic tank
6. conduit
7. cone-shaped tubular adapter
8. assembled instrument unit with the conduit and hydraulic injection device
9. opening for the joint of conduit at the connection of tubes and cone-shaped tubular adapter
10. edge of the internal tube
11. 15° angle cut in at the inner wall of the internal tube
12. last different color band mounted on the internal tube
13. inner pushing part of the hydraulic injector
14. blocking straws on the inner pushing part of the hydraulic injector
15. end of outer tube of the hydraulic injector
16. outer tube of the hydraulic injector
17. outlet of the outer tube of the hydraulic injector
18. groove on cone-shaped tubular adapter to fit in conduit
19. cut at the opening end of the cone-shaped tubular adapter
20. lock bracket on the cone-shaped tubular adapter for the conduit to fit in
21. outer edge of the opening end of cone-shaped tubular adapter
22. liquid as the media driving force inside of the hydraulic tank and the conduit
23. lesions of human organ
24. insertion end of the endoscope
25. direction of driving force forward
26. due directions of driving force forward and backward
27. fillister on the top of the piston
28. dovetail on the side of the piston
29. joint of the conduit to the hydraulic injector
30. standard endoscope used in the market
31. ligating positionor
32. receiver of the infrared sensor
33. sender of the infrared sensor
34. cable from the ligating positioner to the control circuit
35. angle showing the position of the head of the instrument when ligating positioner is applied
36. control panel of the hydraulic injection device
37. cable to connect the controlling panel and control circuit
38. control key pat, 45 control circuit
39. hydraulic injection device
40. actuating motor
41. pushrod
42. positioning adjusting pole
43. positioning adjustor
44. groove to position the hydraulic injentor
45. control circuit of the hydraulic injection device
46. stand for support the hydraulic injector
47. return botton
48. positioning button
49. reset memory button
50. motor button
51. dislodging button
52. buzzer
53. digital screen
54. cylindrical screw for pushrod
55. positioning sloth on the glove of the pushrod
56. pitch
57. connector of hydraulic injector

The patents cited in the present specification including all those mentioned and cited as prior art in their specification, published patent applications as well as all their foreign counterparts and all cited references therein are incorporated in their entirety by reference herein as if those references were denoted in the text.

While the many forms of the invention herein disclosed constitute presently preferred embodiments, many others are possible and further details of the preferred embodiments and other possible embodiments are not to be construed as limitations. It is understood that the terms used herein are merely descriptive rather than limiting and that various changes and many equivalents may be made without departing from the spirit or scope of the claimed invention.

What is claimed is:

1. A hydraulically operated ligating band dispenser for use with an endoscopic device, said dispenser being adapted for supporting a given plurality of elastic ligating bands and comprising:
(a) a double-tubed instrument having two separate tubes with bands and a piston next to the last band mounted on said internal tube, said internal tube having bands and piston placed inside of the external tube;
(b) a flexible cone-shaped tubular adapter for securing both tubes and creating a tubular instrument with one end for connection to the inserting end of an endoscope;
(c) a freely moving piston between the tubes which secures completely a sealed space from the cone-shaped tubular adapter creating a hydraulic tank between the piston and the cone-shaped tubular adapter;
(d) conduit means for conveying liquid serving as the driving force to move said bands forward and dislodge the them one at a time to effect ligation of multiple lesions during a single insertion of the endoscope.

2. The dispensing device of claim 1 wherein said conveying liquid is selected from the group consisting of purified water, sterilized water and physiological saline.
